(19) United States
(12) Patent Application Publication
(10) Pub. No.: US 2005/0182097 A1
(43) Pub. Date: Aug. 18, 2005

(54) Methods and compositions using thalidomide for the treatment and management of central nervous system disorders or diseases

(76) Inventors: Jerome B. Zeldis, Princeton, NJ (US); Herbert Faleck, West Orange, NJ (US); Peter H. Schafer, Somerset, NJ (US)

Correspondence Address: JONES DAY, 222 EAST 41ST ST, NEW YORK, NY, 10017, US

(21) Appl. No.: 11/022,065

(22) Filed: Dec. 23, 2004

Related U.S. Application Data
(60) Provisional application No. 60/533,864, filed on Dec. 30, 2003.

(51) Int. Cl. A61K 31/454

(52) U.S. Cl. 514/323

(57) ABSTRACT

Methods of treating, preventing and/or managing central nervous system disorders, such as Amyotrophic Lateral Sclerosis (ALS or Lou Gehrig's Disease) and related syndromes are disclosed. Specific methods encompass the administration of thalidomide, or a pharmaceutically acceptable salt, solvate, hydrate, or stereoisomer thereof, alone or in combination with a second active ingredient. Pharmaceutical compositions, single unit dosage forms, and kits suitable for use in methods of the invention are also disclosed.

This invention claims the benefit of U.S. Provisional Application No. 60/533,864, filed Dec. 30, 2003, which is incorporated herein in its entirety by reference.

### 1. FIELD OF THE INVENTION

This invention relates, in part, to methods of treating, preventing and/or managing central nervous system disorders, including but not limited to, Amyotrophic Lateral Sclerosis (ALS or Lou Gehrig's Disease) and related disorders which comprise the administration of thalidomide, or a pharmaceutically acceptable salt, solvate, hydrate, or stereoisomer thereof, alone or in combination with known therapeutics.

### 2. BACKGROUND OF THE INVENTION

Central nervous system disorders affect a wide range of the population with differing severity. Generally, the major feature of this class of disorders include the significant impairment of cognition or memory that represents a marked deterioration from a previous level of functioning. Dementia, for example, is characterized by several cognitive impairments including significant memory deficit and can stand alone or be an underlying characteristic feature of a variety of diseases, including Alzheimer Disease, Parkinson disease, Huntington's Disease, and Multiple Sclerosis to name but a few. Other central nervous system disorders include delerium, or disturbances in consciousness that occur over a short period of time, and amnestic disorder, or discreet memory impairments that occur in the absence of other central nervous system impairments.

2.1 Thalidomide

Thalidomide is a racemic compound sold under the tradename Thalomid® and chemically named α-(N-phthalimido)glutarimide or 2-(2,6-dioxo-3-piperidinyl)-1H-isoindole-1,3(2H)-dione. The compound has structure I:

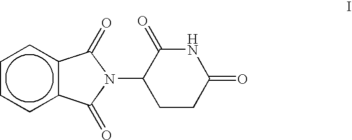

Thalidomide was originally developed in the 1950's to treat morning sickness, but due to its teratogenic effects was withdrawn from use. Thalidomide has been approved in the United States for the acute treatment of the cutaneous manifestations of erythema nodosum leprosum in leprosy. Physicians' Desk Reference, 1153-1157 (57th ed., 2003). Because its administration to pregnant women can cause birth defects, the sale of thalidomide is strictly controlled. Id. Thalidomide has been studied in the treatment of other diseases, such as chronic graft-vs-host disease, rheumatoid arthritis, sarcoidosis, several inflammatory skin diseases, and inflammatory bowel disease. See generally, Koch, H. P., Prog. Med. Chem. 22:165-242 (1985). See also, Moller, D. R., et al., J. Immunol. 159:5157-5161 (1997); Vasiliauskas, E. A., et al., Gastroenterology 117:1278-1287 (1999); Ehrenpreis, E. D., et al., Gastroenterology 117:1271-1277 (1999). It has further been alleged that thalidomide can be combined with other drugs to treat ischemia/repercussion associated with coronary and cerebral occlusion. See U.S. Pat. No. 5,643,915, which is incorporated herein by reference.

Thalidomide was found to exert immunomodulatory and anti-inflammatory effects in a variety of disease states, cachexia in AIDS, and opportunistic infections in AIDS. In studies to define the physiological targets of thalidomide, the drug was found to have a wide variety of biological activities exclusive of its sedative effect including neurotoxicity, teratogenicity, suppression of TNF-α production by monocytes/macrophages and the accompanying inflammatory toxicities associated with high levels of TNF-α, and inhibition of angiogenesis and neovascularization.

Additionally, beneficial effects have been observed in a variety of dermatological conditions, ulcerative colitis, Crohn's disease, Behcets's syndrome, systemic lupus erythematosis, aphthous ulcers, cancer and lupus. The anti-angiogenic properties of thalidomide in in vivo models have been reported. D'Amato et al., Thalidomide Is An Inhibitor Of Angiogenesis, 1994, PNAS, USA 91:4082-4085. Thalidomide also has a palliative effect for a variety of neuropathic states such as Complex Regional Pain Syndrome and radiculopathy.

However, the full spectrum of activity of thalidomide is not fully characterized. Available data from in vitro studies and preliminary clinical trials suggest that the immunologic effects of the compound can vary substantially under different conditions. They may be related to suppression of tumor necrosis factor-alpha (TNF-α) production and downmodulation of selected cell surface adhesion molecules involved in leukocyte migration.

2.2 Amyotrophic Lateral Sclerosis

Amyotrophic Lateral Sclerosis (ALS), commonly known as Lou Gehrig's Disease in the United States, is a neurodegenerative disorder that affects the upper and lower motor neurons resulting in the wasting away of muscles that have lost their innervation. Nature, 1993, 364(6435) 362. As motor neurons degenerate, they can no longer send impulses to the muscle fibers that normally result in muscle movement. ALS usually develops in humans between the ages of 40 and 70. Early symptoms of ALS often include progressive muscle weakness, especially involving the arms and legs, speech, swallowing and breathing. Likewise, ALS can cause slurred speech and difficulty breathing. Pathological characteristics include anterior nerve root shrinkage in addition to spinal cord atrophy. Brain Res. Bull., 1993, 30(3-4), 359-64.

There are three classifications of ALS: Sporadic ALS which represents 90-95% of all ALS cases; Familial ALS which occurs more than once in a family lineage and accounts for 5 to 10% of all cases; and Guamanian ALS, representing an extremely high incidence of ALS observed in Guam and the Trust Territories of the Pacific in the 1950's. ALS typically causes total paralysis and respiratory failure within five years of onset. 50% of ALS patients die within eighteen months after diagnosis.

At present, riluzole (Rilutek™), a glutamate inhibitor, is the only approved therapyfor ALS, and no other therapies for ALS, and no agents are consistently effective in preventing the progression of the disease. The majority of therapeutics that are in current use focus on the management of the symptoms of ALS. However, due to the side effects and unattractive dosing requirements of these drugs, new methods and compounds that are able to treat ALS and its symptoms are highly desirable.

2.3 Parkinson Disease

Parkinson Disease (PD) is the second most common neurodegenerative disease and affects approximately 1% of the population over 50 years of age. Polymeropoulos et. al., 1996, Science 274: 1197-1198. Approximately one million Americans suffer from PD, and each year 50,000 individuals are diagnosed with the disorder. Olson, L., 2000, Science 290:721-724. Because early symptoms of PD may go unrecognized, perhaps as many as 5 to 10% of individuals over 60 years of age may have the illness. Olson, L., 2000, Science 290:721-724.

It has been known since the 1960s that loss of dopamine neurons in the nigrostriatal pathway of the brain results in the motor abnormalities characteristic of PD. Typical onset of PD occurs in mid to late adulthood with progressive clinical features. Some of the physical manifestations of PD include resting tremors, muscular rigidity, postural instability, and dementia. Pathologic characteristics of PD include a loss of dopaminergic neurons in the substantia nigra (SN) as well as the presence of intracellular inclusions or Lewy Bodies in surviving neurons in various areas of the brain. Nussbaum, R. L. and Polymeropoulos, M. H., 1997, Hum. Molec. Genet. 6: 1687-1691. Interestingly, many other diseases have parkisonian motor features. The motor symptoms in PD are generally thought to result from the deficiency or dysfunction of dopamine or dopaminergic neurons in the substantia nigra. Nussbaum, R. L., Polymeropoulos, M. H., 1997, Hum. Molec. Genet. 6: 1687-1691. Evidence has also suggested that molecular chaperones, specifically heat shock proteins, HSP70 and HSP40, may play a role in PD progression. Auluck et. al., 2002, Science 295: 865-868.

Much controversy exists regarding the etiology of PD, and there is evidence that both genetic and environmental factors may contribute to the disease. A study of the nuclear families of 948 PD cases concluded that a rare major mendelian inheritance gene, that influences age of onset, exists. Maher et. al., 2002, Am. J. Med. Genet. 109: 191-197. This study also suggested the existence of a gene that influences susceptibility. Other evidence also suggests that environmental factors may be more significant than genetic factors in contributing to PD. Calne et. al., 1987, Canad. J. Neurol. Sci. 14: 303-305. Researchers have concluded that most cases of PD are caused by environmental factors superimposed on a background of slow and sustained neuronal loss due to aging. Calne, D. B. and Langston, J. W., 1993, Lancet II: 1457-1459. While the etiology remains unclear, it is likely that both genetic and environmental factors contribute to PD, and that environmental factors act upon genetic susceptibility to cause the disease. Recent evidence in animal models of Parkinson disease, suggests that anti-inflammatory agents inhibit dopaminergic cell death. McGeer et. al., 2001, B.C. Med. J. 43:138-141.

While a cure is not currently available for Parkinson disease, traditional treatment has focused on responding to the effect of dopamine loss in the brain. Therapy using dopamine precursor, levodopa, became the treatment of choice when it was discovered that the compound could alleviate PD symptoms, thereby improving the quality of life for affected individuals. Unfortunately, it has become clear that long-term levodopa administration can have side affects. Caraceni et. al., 1994 Neurology, 41:380. A variety of therapeutic strategies have been developed for the treatment of PD. MPTP, a neurotoxin known to specifically damage dopamine neurons, is commonly used as a model for the effects of PD. In one study, investigators used lentiviral vectors to deliver glial cell line derived neurotrophic factor (GDNF) to the striatum and SN of rhesus monkeys that had been treated one week prior with MPTP. Kordower et. al., 2000, Science 290: 767-773. GDNF is known to have trophic effects upon degenerating nigrostriatal neurons in nonhuman primate models of Parkinson disease. Results of the study showed that GDNF augmented dopaminergic function in aged monkeys and reversed functional deficits and prevented nigrostriatal degeneration in monkeys that had been treated with MPTP. It was also noted that GDNF treatment reversed motor deficits in MPTP treated monkeys. This study also concluded that GDNF delivery could prevent nigrostriatal degeneration and induce regeneration of neurons in primate models of PD. Kordower et. al., 2000, Science 290: 767-773.

Another study, using electrical inhibition and pharmacologic silencing of the subthalmic nucleus (STN), demonstrated that the alteration of basal ganglia network activity could improve motor network activity in PD, presumably by suppressing the firing activity of neurons in the SN. Luo et. al., 2002, Science 298: 425-429. Investigators used an adeno-associated virus to transduce excitatory glutaminergic neurons in the rat STN with glutamic acid decarboxylase (GAD) to demonstrate that the change provided neuroprotection to the dopaminergic cells from toxic insults. Interestingly, rats with the transduced gene also showed significant improvement from parkinsonian phenotypes.

2.4 Alzheimer Disease

Alzheimer disease (AD) is an increasingly prevalent form of neurodegeneration that accounts for approximately 50%-60% of the overall cases of dementia among people over 65 years of age. It currently affects an estimated 15 million people worldwide and owing to the relative increase of elderly people in the population its prevalence is likely to increase over the next 2 to 3 decades. Alzheimer disease is a progressive disorder with a mean duration of around 8.5 years between onset of clinical symptoms and death. Death of pyramidal neurons and loss of neuronal synapses in brains regions associated with higher mental functions results in the typical symptoms, characterized by gross and progressive impairment of cognitive function (Francis et al., 1999, J. Neurol. Neurosurg. Psychiatry 66:137-47). Alzheimer disease is the most common form of both senile and presenile dementia in the world and is recognized clinically as relentlessly progressive dementia that presents with increasing loss of memory, intellectual function and disturbances in speech (Merritt, 1979, A Textbook of Neurology, 6th edition, pp. 484-489 Lea & Febiger, Philadelphia). The disease itself usually has a slow and insidious progress that affects both sexes equally, worldwide. It begins with mildly inappropriate behavior, uncritical statements, irritability, a tendency towards grandiosity, euphoria and deteriorating performance at work; it progresses through deterioration in operational judgment, loss of insight, depression and loss of recent memory; it ends in severe disorientation and confusion, apraxia of gait, generalized rigidity and incontinence (Gilroy & Meyer, 1979, Medical Neurology, pp. 175-179 MacMillan Publishing Co.).

The etiology of Alzheimer disease is unknown. Evidence for a genetic contribution comes from several important observations such as the familial incidence, pedigree analysis, monozygotic and dizygotic twin studies and the association of the disease with Down's syndrome (for review see Baraitser, 1990, The Genetics of Neurological Disorders, 2nd edition, pp. 85-88). Nevertheless, this evidence is far from definitive and it is clear that one or more other factors are also required. Elevated concentrations of aluminum have been found in the brains of some patients dying with Alzheimer disease (Crapper et al., 1976, Brain, 99:67-80) and one case report has documented markedly elevated levels of manganese in the tissues of a patient with Alzheimer disease (Banta & Markesberg, 1977, Neurology, 27:213-216), which has led to the suggestion that high levels of these metals may be neurotoxic and lead to the development of Alzheimer disease. It was interesting that the aluminum ions were found to be associated mainly with the nuclear chromatin in brain regions most likely to display neurofibrillary tangles in Alzheimer disease. However, from a statistical point of view the absolute differences found for the aluminum levels between normal and Alzheimer brains were far from convincing. It has recently been suggested that defects in the transcriptional splicing of mRNA coding for the tau complex of microtubule associated proteins occur (for review see Kosik, 1990, Curr. Opinion Cell Biol., 2:101-104) and/or that inappropriate phosphorylation of these proteins exists (Grundke-Igbak et al., 1986, Proc. Natl. Acad. Sci. USA, 83:4913-4917; Wolozin & Davies, 1987, Ann. Neurol. 22:521-526; Hyman et al., 1988, Ann. Neurol., 23:371-379; Bancher et al., 1989, Brain Res., 477:90-99). Furthermore, reduction in the enzymes involved in the synthesis of acetylcholine has led to the view of Alzheimer disease as a cholinergic system failure (Danes & Moloney, 1976, Lancet, ii: 1403-14). However, even if cholinergic neurons are most at risk in Alzheimer disease, it appears likely that these reductions in enzyme activity are secondary to the degenerative process itself rather than causally related.

At present, there are no proven therapies for Alzheimer disease, and no agents are consistently effective in preventing the progression of the disease. The majority of therapeutics that are in current use focus on the management of the symptoms of AD. These strategies have employed the use of anti-psychiatric drugs as well as neuroleptic agents and acetylcholinesterase inhibitors. However, due to the side effects and unattractive dosing requirements of these drugs, new methods and compounds that are able to treat AD and its symptoms are highly desirable.

### 3. SUMMARY OF THE INVENTION

This invention encompasses methods of treating or preventing central nervous system disorders and related disorders which comprise administering to a patient in need of such treatment or prevention a therapeutically or prophylactically effective amount of thalidomide, or a pharmaceutically acceptable salt, solvate, hydrate, or stereoisomer thereof. Central nervous system disorders include, but are not limited to, Amyotrophic Lateral Sclerosis, Alzheimer Disease, Parkinson disease, Huntington's Disease, Multiple Sclerosis other neuroimmunological disorders such as Tourette Syndrome, delerium, or disturbances in consciousness that occur over a short period of time, and amnestic disorder, or discreet memory impairments that occur in the absence of other central nervous system impairments. The invention also encompasses methods of managing neurodegenerative central nervous system disorders (e.g., lengthening the time of remission of their symptoms) which comprise administering to a patient in need of such management, a prophylactically effective amount of thalidomide, or a pharmaceutically acceptable salt, solvate, hydrate, or stereoisomer thereof. Each of these methods includes specific dosing or dosing regimens including cycling therapy.

The invention further encompasses pharmaceutical compositions, single unit dosage forms, and kits suitable for use in treating, preventing and/or managing central nervous system disorders, preferably ALS, which comprise thalidomide, or a pharmaceutically acceptable salt, solvate, hydrate, or stereoisomer thereof.

In particular embodiments of the invention, thalidomide is used, administered, or formulated with one or more second active ingredients to treat, prevent or manage central nervous system disorders, preferably ALS. Examples of the second active ingredients include but are not limited to dopamine agonists, Levodopa, compounds used to augment Levodopa therapy such as monoamine oxidase inhibitors (MAO) and catechol-O-methyltransferase inhibitors (COMT), amantadine, cholinesterase inhibitors, glutamine inhibitors, anticholinergics anticholinergics, antiemetics, and other standard therapies for central nervous system disorders. In another example, the second active ingredients are anti-inflammatory agents, including, but not limited to, nonsteroidal anti-inflammatory drugs (NSAIDs), PDE-4 inhibitors, Jun N terminal kinase inhibitors, Methotrexate, Leflunomide, antimalarial drugs and sulfasalazine, gold salts, glucocorticoids, immunosuppresive agents, and other standard therapies for Parkinson disease and related disorders.

### 4. DETAILED DESCRIPTION OF THE INVENTION

A first embodiment of the invention encompasses methods of treating or preventing a central nervous system disorder, which comprises ALS, which comprises administering to a patient in need of such treatment or prevention a therapeutically or prophylactically effective amount of thalidomide, or a pharmaceutically acceptable salt, solvate, hydrate, or stereoisomer thereof. Central nervous system disorders, include, but are not limited to, Amyotrophic Lateral Sclerosis (ALS), Parkinson disease; bradykinesia; muscle rigidity; parkinsonian tremor; parkinsonian gait; motion freezing; depression; dementia; sleep disorders; postural instability; hypokinetic disorders; CNS and peripheral nerve inflammation; synuclein disorders; multiple system artrophies; striatonigral degeneration; olivopontocerebellar atrophy; Shy-Drager syndrome; motor neuron disease with parkinsonian features; Lewy body dementia; Tau pathology disorders; progressive supranculear palsy; corticobasal degeneration; frontotemporal dementia; amyloid pathology disorders; alzheimer disease; alzheimer disease with parkinsonism; genetic disorders that can have parkinsonian features; Wilson disease; Hallervorden-Spatz disease; Chediak-Hagashi disease; SCA-3 spinocerebellar ataxia; X-linked dystonia parkinsonism; Huntington disease; prion disease; hyperkinetic disorders; chorea; ballismus; dystonia tremors; tic disorders including but not limited to Tourette Syndrome; CNS trauma and myoclonus. A specific central nervous system disorder is Amyotrophic Lateral Sclerosis.

Another embodiment of the invention encompasses methods of managing a central nervous system disorder, which comprises administering to a patient in need of such management a prophylactically effective amount of thalidomide, or a pharmaceutically acceptable salt, solvate, hydrate, or stereoisomer thereof.

Another embodiment of the invention encompasses a method of treating, preventing and/or managing a central nervous system disorder, which comprises administering to a patient in need of such treatment, prevention and/or management a therapeutically or prophylactically effective amount of thalidomide, or a pharmaceutically acceptable salt, solvate, hydrate, or stereoisomer thereof and a therapeutically or prophylactically effective amount of a second active agent.

Another embodiment of the invention encompasses a method of reversing, reducing or avoiding an adverse effect associated with the administration of conventional therapy for central nervous system disorders to a patient suffering from central nervous system disorders or a related disorder, which comprises administering to a patient in need of such reversion, reduction or avoidance a therapeutically or prophylactically effective amount of thalidomide, or a pharmaceutically acceptable salt, solvate, hydrate, or stereoisomer thereof.

Yet another embodiment of the invention encompasses a pharmaceutical composition comprising thalidomide, or a pharmaceutically acceptable salt, solvate, hydrate, or stereoisomer thereof, and a pharmaceutically acceptable carrier, diluent or excipient wherein the composition is adapted for parenteral, oral or transdermal administration and the amount is sufficient to treat or prevent a central nervous system disorder, preferably ALS or to ameliorate the symptoms or progress of the disease.

Also encompassed by the invention are single unit dosage forms comprising thalidomide, or a pharmaceutically acceptable salt, solvate, hydrate, or stereoisomer thereof.

Second active agents can be large molecules (e.g., proteins) or small molecules (e.g., synthetic inorganic, organometallic, or organic molecules). The examples of the second active agent include, but are not limited to, cytokines, hematopoietic growth factors, anti-cancer agents such as topoisomerase inhibitors, anti-angiogenic agents, microtubule stabilizing agents, apoptosis inducing agents, alkylating agents and other conventional chemotherapy described in the Physician's Desk Reference 2002; cholinesterate inhibitors; antivirals; antifungals; antibiotics; anti-inflammatories; immunomodulatory agents; immunosuppressive agents such as cyclosporins; and other known or conventional agents used in ALS, or Parkinson disease patients. Specific second active agents include but are not limited to riluzole for ALS, a dopamine agonist or antagonist for Parkinson disease or a cholinesterate inhibitor for Alzheimer Disease.

The invention also encompasses kits which comprise thalidomide, or a pharmaceutically acceptable salt, solvate, hydrate, or stereoisomer thereof, a second active ingredient.

Thalidomide can either be commercially purchased from Celgene Corporation (Warren, N.J.) or prepared according to the well-known methods. Salts, solvates and hydrates of thalidomide can be synthesized using methods well-known to the skilled practicioner. Further, optically pure compositions can be asymmetrically synthesized or resolved using known resolving agents or chiral columns as well as other standard synthetic organic chemistry techniques. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N.Y., 1962); and Wilen, S. H., Tables of Resolving Agents and Optical Resolutions p. 268 (E.L. Eliel, Ed., Univ. of Notre Dame Press, Notre Dame, IN, 1972).

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

Thalidomide contains one or more chiral centers, and can exist as racemic mixtures of enantiomers or mixtures of diastereomers. This invention encompasses the use of stereomerically pure forms of thalidomide, as well as the use of mixtures of those forms. For example, mixtures comprising equal or unequal amounts of the enantiomers of thalidomide may be used in methods and compositions of the invention. The purified (R) or (S) enantiomers of the specific compounds disclosed herein may be used substantially free of its other enantiomer.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “enantiomerically pure” means a stereomerically pure composition of a compound having one chiral center.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

4.1 Second Active Ingredients

As discussed above, a second active ingredient or agent can be used in the methods and compositions of the invention together with thalidomide, particularly conventional agents or therapies used to treat or manage central nervous system disorders. Specific second active agents also stimulate the division and differentiation of committed erythroid progenitors in cells in vitro or in vivo.

In one embodiment, a second active ingredient can be administered with thalidomide, or a pharmaceutically acceptable salt, solvate, hydrate, or stereoisomer thereof. In a specific embodiment, the second active ingredient is riluzole. In one embodiment, the second active ingredient is a dopamine agonist or antagonist, for example, but not limited to, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, or Symmetrel.

In another embodiment, the second active ingredient that is administered with thalidomide, or a pharmaceutically acceptable salt, solvate, hydrate, or stereoisomer thereof is a MAO inhibitor, for example, but not limited to, iproniazid, clorgyline, phenelzine and isocarboxazid.

In another embodiment, the second active ingredient that is administered with thalidomide, or a pharmaceutically acceptable salt, solvate, hydrate, or stereoisomer thereof is a COMT inhibitor, for example, but not limited to, tolcapone and entacapone.

In another embodiment, the second active ingredient that is administered with thalidomide, or a pharmaceutically acceptable salt, solvate, hydrate, or stereoisomer thereof is a cholinesterase inhibitor, for example, but not limited to, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, and demecarium.

In yet another emodiment, the second active ingredient that is administered with thalidomide, or a pharmaceutically acceptable salt, solvate, hydrate, or stereoisomer thereof is an anti-inflammatory agent, including, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHₒ-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids.

In even another embodiment, the second active ingredient that is administered with thalidomide, or a pharmaceutically acceptable salt, solvate, hydrate, or stereoisomer thereof is an antiemetic agent, for example, but not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, and mixtures thereof.

4.2 Methods of Treatment and Management

Methods of this invention encompass methods of preventing, treating and/or managing central nervous system disorders, preferably ALS, Parkinson disease, neuroimmunological disorders such as Tourette Syndrome or Alzheimer Disease. As used herein, unless otherwise specified, the term “preventing” includes but is not limited to, inhibition or the averting of symptoms associated with neurodegenerative central nervous system disorders. Central nervous system disorders, include, but are not limited to, Amyotrophic Lateral Sclerosis (ALS); progressive motor deterioration; CNS trauma; hypokinetic disorders; bradykinesia; slowness of movement; paucity of movement; impairment of dexterity; hypophonia; monotonic speech; muscular rigidity; masked faces; decreased blinking; stooped posture; decreased arm swinging when walking; micrographia; parkinsonian tremor; parkinsonian gait; postural instability; festinating gait; motion freezing; disturbances of cognition, mood, sensation, sleep or autonomic function; dementia; depression and sleep disorders. As used herein, unless otherwise specified, the term “treating” refers to the administration of a composition after the onset of symptoms of central nervous system disorders, preferably Parkinson disease or a related disorder whereas “preventing” refers to the administration prior to the onset of symptoms, particularly to patients at risk of central nervous system disorders, preferably Parkinson disease or a related disorder. As used herein and unless otherwise indicated, the term “managing” encompasses preventing the recurrence of symptoms of central nervous system disorders in a patient who had suffered from a central nervous system disorder, lengthening the time the symptoms remain in remission in a patient who had suffered from central nervous system disorders, and/or preventing the occurrence of central nervous system disorders in patients at risk of suffering from central nervous system disorders.

In a specific embodiment, the central nervous system disorder to be prevented, treated and/or managed is not Parkinson disease, but is Alzheimer Disease, dementia, depression, Alamyotrophic Lateral Sclerosis (ALS), neuroimmunological disorders or CNS trauma.

The invention encompasses methods of treating or preventing central nervous system disorders, preferably ALS, Parkinson disease or Azlheimer disease. In one embodiment, the methods of the invention are used to treat or prevent disorders related to movement, including, but not limited to, progressive motor deterioration, slow execution or bradykinesia, paucity of movement or akinesia, movement disorders that impair fine motor control and fmger dexterity, and other manifestations of bradykinesia, such as, but not limited to, hypophonia and monotonic speech. In another embodiment, the methods of the invention are used to treat or prevent disorders related to muscular rigidity, including, but not limited to, a uniform increase in resistance to passive movement, interruptions to passive movement, and combinations of rigidity and dystonia. In a specific embodiment, methods of the invention are used to treat inflammation associated with Parkinsonor related disease. In yet another embodiment of the invention, disorders resembling Parkinsonian tremor are treated or prevented by the methods of the invention, including but not limited to, tremors of the face, jaw, tongue, posture, and other tremors that are present at rest and that attenuate during movement. In another embodiment, the methods of the invention are used to treat or prevent disorders in gait, including, but not limited to, those resembling parkinsonian gait, shuffling, short steps, a tendency to turn en bloc, and festinating gait. In another embodiment of the invention, nonmotor symptoms are treated or prevented using the methods of the invention, including, but not limited to, disorders of mood, cognition, sensation, sleep, dementia, and depression. In other embodiment of the invention secondary forms of parkinsonism are treated or prevented by the methods of the invention, including, but not limited to, drug induced parkinsonism, vascular parkinsonism, multiple system atrophy, progressive supranuclear palsy, disorders with primary tau pathology, cortical basal ganglia degeneration, parkinsonism with dementia, hyperkinetic disorders, chorea, Huntington's disease, dystonia, Wilson disease, Tourette syndrome, essential tremor, myoclonus, and tardive movement disorders. In other embodiment of the invention other central nervous system disorders are treated or prevented by the methods of the invention, including, but not limited to Alzheimer Disease, Amyotrophic Lateral Sclerosis (ALS) and CNS trauma.

Methods encompassed by this invention comprise administering thalidomide, or a pharmaceutically acceptable salt, solvate, hydrate, or stereoisomer thereof to a patient (e.g., a human) suffering, or likely to suffer, from central nervous system disorders.

Another method comprises administering 1) thalidomide, or a pharmaceutically acceptable salt, solvate, hydrate, or stereoisomer thereof, and 2) a second active agent or active ingredient. Examples of examples of the second active agents are also disclosed herein (see, e.g., section 4.2).

Administration of thalidomide and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for thaliomide is orally. Preferred routes of administration for the second active agents or ingredients of the invention are known to those of ordinary skill in the art. See, e.g., Physicians' Desk Reference, 1755-1760 (56^{th} ed., 2002).

In one embodiment of the invention, the recommended daily dose range of thalidomide for the conditions described herein lie within the range of from about 1 mg to about 10,000 mg per day, given as a single once-a-day dose, or preferably in divided doses throughout a day. More specifically, the daily dose is administered twice daily in equally divided doses. Specifically, a daily dose range should be from about 1 mg to about 5,000 mg per day, more specifically, between about 10 mg and about 2,500 mg per day, between about 100 mg and about 800 mg per day, between about 100 mg and about 1,200 mg per day, or between about 25 mg and about 2,500 mg per day. In managing the patient, the therapy should be initiated at a lower dose, perhaps about 1 mg to about 2,500 mg, and increased if necessary up to about 200 mg to about 5,000 mg per day as either a single dose or divided doses, depending on the patient's global response. In a particular embodiment, thalidomide can be preferably administered in an amount of about 400, 800, 1,200, 2,500, 5,000 or 10,000 mg a day as two divided doses.

In another embodiment, thalidomide is administered in conjunction with the second active agent. The second active agent is administered orally, intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the disorder being treated or managed, the severity and stage of the central nervous system disorder, and the amount(s) of thalidomide and any optional additional active agents concurrently administered to the patient.

In certain embodiments, the prophylactic or therapeutic agents of the invention are cyclically administered to a patient. Cycling therapy involves the administration of a first agent for a period of time, followed by the administration of the agent and/or the second agent for a period of time and repeating this sequential administration. Cycling therapy can reduce the development of resistance to one or more of the therapies, avoid or reduce the side effects of one of the therapies, and/or improves the efficacy of the treatment.

In a preferred embodiment, prophylactic or therapeutic agents are administered in a cycle of about 24 weeks, about once or twice every day. One cycle can comprise the administration of a therapeutic or prophylactic agent and at least one (1) or three (3) weeks of rest. The number of cycles administered is from about 1 to about 12 cycles, more typically from about 2 to about 10 cycles, and more typically from about 2 to about 8 cycles.

4.3 Pharmaceutical Compositions and Single Unit Dosage Forms

Pharmaceutical compositions can be used in the preparation of individual, single unit dosage forms. Pharmaceutical compositions and dosage forms of the invention comprise a thalidomide, or a pharmaceutically acceptable salt, solvate, hydrate, or stereoisomer thereof. Pharmaceutical compositions and dosage forms of the invention can further comprise one or more excipients.

Pharmaceutical compositions and dosage forms of the invention can also comprise one or more additional active ingredients. Consequently, pharmaceutical compositions and dosage forms of the invention comprise the active ingredients disclosed herein (e.g., thalidomide, or a pharmaceutically acceptable salt, solvate, hydrate, or stereoisomer thereof, and a second active ingredient). Examples of optional additional active ingredients are disclosed herein (see, e.g., section 4.2).

Single unit dosage forms of the invention are suitable for oral, mucosal (e.g., nasal, sublingual, vaginal, buccal, or rectal), or parenteral (e.g., subcutaneous, intravenous, bolus injection, intramuscular, or intraarterial), transdermal or transcutaneous administration to a patent. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Phannaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise thalidomide, or a pharmaceutically acceptable salt, solvate, hydrate, or stereoisomer thereof in an amount of from about 1 to about 1,200 mg. Typical dosage forms comprise thalidomide, or a pharmaceutically acceptable salt, solvate, hydrate, or stereoisomer thereof in an amount of about 1, 2, 5, 10, 25, 50, 100, 200, 400, 800, 1,200, 2,500, 5,000 or 10,000 mg. In a particular embodiment, a preferred dosage form comprises thalidomide in an amount of about 400, 800 or 1,200 mg. Typical dosage forms comprise the second active ingredient in an amount of 1 to about 1000 mg, from about 5 to about 500 mg, from about 10 to about 350 mg, or from about 50 to about 200 mg. Of course, the specific amount of the second active ingredient will depend on the specific agent used, the disorder being treated or managed, and the amount(s) of thalidomide and any optional additional active agents concurrently administered to the patient.

4.3.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Phannaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa,), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, MD), a coagulated aerosol of synthetic silica (marketed by Degussa Co. of Plano, Tex.), CAB-O-SWL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises thalidomide, anhydrous lactose, microcrystalline cellulose, polyvinylpyrrolidone, stearic acid, colloidal anhydrous silica, and gelatin.

4.3.2 Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.3.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of thalidomide. See, e.g., U.S. Pat. No. 5,134,127, which is incorporated herein by reference.

4.3.4 Topical and Mucosal Dosage Forms

Topical and mucosal dosage forms of the invention include, but are not limited to, sprays, aerosols, solutions, emulsions, suspensions, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.3.5 Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a dosage form of thalidomide, or a pharmaceutically acceptable salt, solvate, hydrate, or stereoisomer thereof. Kits encompassed by this invention can further comprise additional active ingredients. Examples of the additional active ingredients include, but are not limited to, those disclosed herein (see, e.g., section 4.2).

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

4.4 Cycling Therapy in Central Nervous System Disorders

In a specific embodiment, thalidomide is cyclically administered to patients with central nervous system disorders. Cycling therapy involves the administration of a first agent for a period of time, followed by the administration of the agent and/or the second agent for a period of time and repeating this sequential administration. Cycling therapy can reduce the development of resistance to one or more of the therapies, avoid or reduce the side effects of one of the therapies, and/or improves the efficacy of the treatment.

In a specific embodiment, prophylactic or therapeutic agents in an amount of about 400, 800 or 1200 mg are administered in a cycle of about 24 weeks, about once or twice every day. One cycle can comprise the administration of a therapeutic on prophylactic agent and at least one (1), two (2), or three (3) weeks of rest. The number of cycles administered is from about 1 to about 12 cycles, more typically from about 2 to about 10 cycles, and more typically from about 2 to about 8 cycles.

### 5. EXAMPLES

The following studies are intended to further illustrate the invention without limiting its scope.

5.1 Studies in Amyotrophic Lateral Sclerosis

The effects of thalidomide in a model of Amyotrophic Lateral Sclerosis are investigated in mice. Thalidomide is administered to Male Transgenic mice overexpressing the human mutated form (G93A) of Cu,Zn-superoxide dismutase (mSOD1) (Science, 302, 113-117, 2003) once or twice daily for 14 days. Anti-ALS activity of thalidomide is assessed by measuring rescue of motoneurons or prolongation of survival in comparison to the reference compound, riluzole.

5.2 Studies in Parkinson Disease

The effects of thalidomide in a model of Parkinson disease are investigated in mice. Male C57/BL6 mice are injected once daily for 7 days with MPTP (30 mg/kg, i.p.). Thalidomide is administered once or twice daily for 14 days. On day 28, striata are removed, homogenized in perchloric acid, and centrifuged. The supernatant is removed and analyzed for dopamine and other monoamines such as serotonin by reverse-phase HPLC and electrochemical detection. Anti-Parkinson activity of thalidomide is assessed in comparison to the reference compound, selegiline.

5.3 Studies in Alzheimer Disease

The effects of thalidomide in a model of Alzheimer disease are investigated in rat PC12 pheochromocytoma cells. PC12 cells are cultured in the presence of dopamine, D1dopamine receptor agonist, adenosine, adenosine A2a receptor agonist, nicotine, or alpha 7 nicotinic acetylcholine receptor agonist and thalidomide. After 24 hours, cellular supernatants are harvested and assayed for acetylcholinesterase activity by the Ellman method (Hawkins and Knittle, Anal Chem 44:416-417,1972). Suppression of acetylcholinesterase activity levels by thalidomide is assessed in comparison to the reference compound tacrine.

5.4 Cycling Therapy in Central Nervous System Disorders

On day 1 in a cycle of 24 weeks, blood product transfusion is administered to patients with ALS. On day 10, the administration of 800 mg/d of thalidomide is started. On day 30, blood product transfusion is administered. On day 34, the administration of 800 mg/d of thalidomide is stopped. On day 59, the administration of 400 mg/d of thalidomide is begun.

Embodiments of the invention described herein are only a sampling of the scope of the invention. The full scope of the invention is better understood with reference to the attached claims.

1. A method of treating or preventing a central nervous system disorder, which comprises administering to a patient in need of such treatment or prevention a therapeutically or prophylactically effective amount of thalidomide, or a pharmaceutically acceptable salt, solvate, hydrate, or stereoisomer thereof.

2. A method of managing a central nervous system disorder, which comprises administering to a patient in need of such management a prophylactically effective amount of thalidomide, or a pharmaceutically acceptable salt, solvate, hydrate, or stereoisomer thereof.

3. The method of claim 1 which comprises administering to a patient in need of such treatment a therapeutically or prophylactically effective amount of thalidomide.

4. The method of claim 2 which comprises administering to a patient in need of such management a prophylactically effective amount of thalidomide.

5. The method of claim 1 wherein the central nervous system disorder is Parkinson disease; Alzheimer disease; Amyotrophic Lateral Sclerosis; progressive motor weakness; neuroimmunological disorders; CNS trauma; Alzheimer disease with parkinsonism; bradykinesia; alkinesia; movement disorders that impair fme motor control and finger dexterity; hypophonia; monotonic speech; rigidity; dystonia; inflammation associated with Parkinson disease; tremors of the face, jaw, tongue, posture; parkinsonian gait; shuffling; short steps; festinating gait; disorders of mood, cognition, sensation, sleep; dementia; depression; drug induced parkinsonism; vascular parkinsonism; multiple system atrophy; progressive supranuclear palsy; disorders with primary tau pathology; cortical basal ganglia degeneration; parkinsonism with dementia; hyperkinetic disorders; chorea; Huntington's disease; dystonia; Wilson disease; Tourette syndrome; essential tremor; myoclonus; or a tardive movement disorder.

6. The method of claim 2 wherein the central nervous system disorder is Parkinson disease; Alzheimer disease; Amyotrophic Lateral Sclerosis; progressive motor weakness; neuroimmunological disorders; CNS trauma; Alzheimer disease with parkinsonism; bradykinesia; alkinesia; movement disorders that impair fine motor control and finger dexterity; hypophonia; monotonic speech; rigidity; dystonia; inflammation associated with Parkinson disease; tremors of the face, jaw, tongue, posture; parkinsonian gait; shuffling; short steps; festinating gait; disorders of mood, cognition, sensation, sleep; dementia; depression; drug induced parkinsonism; vascular parkinsonism; multiple system atrophy; progressive supranuclear palsy; disorders with primary tau pathology; cortical basal ganglia degeneration; parkinsonism with dementia; hyperkinetic disorders; chorea; Huntington's disease; dystonia; Wilson disease; Tourette syndrome; essential tremor; myoclonus; or a tardive movement disorder.

7. The method of claim 5 wherein the central nervous system disorder is Amyotrophic Lateral Sclerosis.

8. The method of claim 6 wherein the central nervous system disorder is Amyotrophic Lateral Sclerosis.

9. A method of treating or preventing a central nervous system disorder, which comprises administering to a patient in need of such treatment or prevention a therapeutically or prophylactically effective amount of thalidomide, or a pharmaceutically acceptable salt, solvate, hydrate, or stereoisomer thereof, and a therapeutically or prophylactically effective amount of at least one second active ingredient.

10. A method of managing a central nervous system disorder, which comprises administering to a patient in need of such management a prophylactically effective amount of thalidomide, or a pharmaceutically acceptable salt, solvate, hydrate, or stereoisomer thereof, and a therapeutically or prophylactically effective amount of at least one second active ingredient.

11. The method of claim 9 wherein the central nervous system disorder is Parkinson disease.

12. The method of claim 10 wherein the central nervous system disorder is Parkinson disease.

13. The method of claim 9, wherein the second active ingredient is riluzole, a dopamine agonist, a monoamine oxidase inhibitor (MAO), a catechol-O-methyltransferase inhibitor (COMT), amantadine, a cholinesterase inhibitor, an antiemetic, or an anti-inflammatory agent.

14. The method of claim 10, wherein the second active ingredient is riluzole, a dopamine agonist, a monoamine oxidase inhibitor (MAO), a catechol-O-methyltransferase inhibitor (COMT), amantadine, a cholinesterase inhibitor, an antiemetic, or an anti-inflammatory agent.

15. The method of any one of claims 1, 2, 9, or 10, wherein the stereoisomer of thalidomide is the R or S enantiomer of thalidomide.

16. A method of reducing or avoiding an adverse effect associated with the administration of a second active ingredient in a patient suffering from a central nervous system disorder, which comprises administering to a patient in need of such reduction or avoidance an amount of the second active ingredient and a therapeutically or prophylactically effective amount of thalidomide, or a pharmaceutically acceptable salt, solvate, hydrate, or stereoisomer thereof.

17. A pharmaceutical composition comprising thalidomide, or a pharmaceutically acceptable salt, solvate, hydrate, or stereoisomer thereof in an amount effective to treat, prevent or manage a central nervous system disorder, and a carrier.

18. A pharmaceutical composition comprising thalidomide, or a pharmaceutically acceptable salt, solvate, hydrate, or stereoisomer thereof, in an amount effective to treat, prevent or manage a central nervous system disorder, and a second active ingredient.

19. The pharmaceutical composition of claim 18 wherein the second active ingredient is riluzole, a dopamine agonist, a monoamine oxidase inhibitor (MAO), a catechol-O-methyltransferase inhibitor (COMT), amantadine, an anticholinergic, an antiemetic, or an anti-inflammatory agent.
